(19) United States
(12) Patent Application Publication
(10) Pub. No.: US 2005/0074366 A1
(43) Pub. Date: Apr. 7, 2005

(54) Method for determining phase distribution coefficients, in addition to a corresponding device

(76) Inventors: Giovanni D'Orsaneo, Julich (DE); Volker Linnemann, Aachen (DE); Johan Smelt, Andelst (NL); Kilian Eric Christopher Smith, Rostock (DE); Andre Wolters, Koln (DE)

Correspondence Address: THE FIRM OF KARL F ROSS, 5676 RIVERDALE AVENUE, PO BOX 900, RIVERDALE (BRONX), NY, 10471-0900, US

(21) Appl. No.: 10/499,746

(22) PCT Filed: Dec. 12, 2002
(86) PCT No.: PCT/DE02/04550

(30) Foreign Application Priority Data
Dec. 20, 2001 (DE) 101 62 852.8

(51) Int. Cl. G01N 33/00

(52) U.S. Cl. 422/88; 422/68.1

(57) ABSTRACT

The invention relates to a method for determining phase distribution coefficients of substances for the solid/gas and liquid/gas compartments. According to the invention, a gas that has been pre-treated with active charcoal is fed through a housing (1), in which a sample has been provided. If the sample is a solid, the gas enters through openings in a shaft (11), which traverses the housing (1) and is equipped with propellers. If the sample is a liquid, the gas enters through a separate opening (8a, b, c) and the surface area of the liquid is increased by a rotating cylinder (9). The gas emerges from the housing (1) and traverses an adsorption tube, which is subjected to thermodesorption after the experiment. The substance released by said thermodesorption is subjected to a quantitative analysis.

The invention relates to a method of determining phase distribution coefficients of substances for the gas/liquid compartments and gas/solids compartments, especially in the soil-air system and the water-air system, as well as a device suitable for practicing the method.

The Henry coefficient (K_{H}) describes the equilibrium distribution of a substance between the water and air compartment, that is it indicates the ratio of the substance concentration in the air to the substance concentration in water at equilibrium; analogously, the soil-air distribution coefficient describes the equilibrium distribution between the soil and air compartments.

In accordance with the state of the art, different methods for determining distribution coefficients are known.

Various experimental methods for determining Henry constants K_{H} depend on the one hand upon a determination of water concentration and air concentration of the investigated compound in closed systems after attaining an equilibrium state and thus upon the measurements of relative concentration changes in one phase while the other phase in the presence of a quasiequilibrium has reached and is held at the rapidly achieved equilibrium. In the “wetted” wall column method, at low flow rates, an equilibrium is brought about between a water film which flows downwardly in a vertical column and an oppositely flowing gas stream.

The test substance which can be added to the gas stream or to the water phase is analytically quantified in both phases (Fendinger & Glotfelty, Environ, Toxicol. Chem. 1988, 22, 1289-1293). The “gas sparging” method for determining the Henry constant of high molecular weight and halogenated hydrocarbons depends upon the isothermal extraction of the compounds from concentrated aqueous solutions by passing a nitrogen stream through them (Mackay et al., Environ. Sci Technol. 1979, 13, 333-337).

For determining soil-air distributions, dynamic system (“flow through systems”) are used: a purified air stream is thus passed through a column filled with soil so that between the soil phase and the air phase an equilibrium state is established (Hippelein & McLachlan, Environ. Sci Technol. Head space in the chambers through which the air stream can pass after traversing the soil-containing chamber have been used up to now exclusively for determining volatilization rates and enable calculation of the soil-air distribution coefficients by use of equilibrium concentrations in the soil and in the air (Ayris & Harrad, J. Environ. Monit. 1999, 1, 395-401; Morrissey & Grimer, J. Contamin. Hydr. 1999, 36, 291-312).

With the method in accordance with the state of the art and devices for carrying out the method, it has up to now not been possible to measure distribution coefficients of low volatility compounds like, for example, plant protective agents with Henry constants, preferably in the range of 10⁻⁷ to 10⁻⁹.

The analytical detection limits especially pose a problem in characterizing the temperature and soil moisture effects so that to date quantification of these influences has not been possible with sufficient precision.

A problem in the use of dynamic methods resides in the adjustment of the equilibrium, that is that with the use of low flow velocity it cannot be ensured that with low volatility compounds, there will be sufficient contact time between the soil-air or water-air compartments for establishing concentration equilibria. In addition this difficulty is increased in the case of the soil-air system by soil processes in that, for determining the distribution coefficient, it is necessary to establish an equilibrium between the air and the upper soil layer which is in direct contact with the air. A precondition for an equilibrium is however, that the mutual interaction with the air not give rise to an “emptying” of [the compound investigated from] the upper soil layer. The substances losses at the surface must be compensated by diffusion from lower soil layers and then the diffusion process counteracts the possible absorption of the soil. This kinetic problem results in disequilibrium in efforts to measure distribution coefficients which are not constant with time.

Furthermore, up to now, no apparatus has been provided which can permit, because of its structural configuration, both the measurement of the distribution coefficients for water-air as well as for soil-water.

It is thus the object of the invention to provide a method of and a device for determining the phase distribution coefficients of substances in the liquid-gas and solids-gas phase systems, especially, water-air and soil-air. Furthermore, the precision of the method should be great enough that even the distribution coefficient of very low volatility compounds can be determined. These can be, for example, pesticides, PCVs or PAKs.

In addition, the aforementioned process drawbacks should be eliminated.

Starting with the preamble of claim 1, the objects are achieved according to the invention by features in the characterization clause.

With the method and the apparatus of the invention, it is only now possible to determine phase distribution coefficients of substances in the water-air soil-water systems with Henry constants K_{H} of 10⁻⁷ to 10^{-9.}

Advantageously features of the invention are given in the dependent claims.

In the following, the invention is described in greater detail as to examples of the water-air and soil-air compartments.

The Figures show by way of example an embodiment of the device of the invention.

They are:

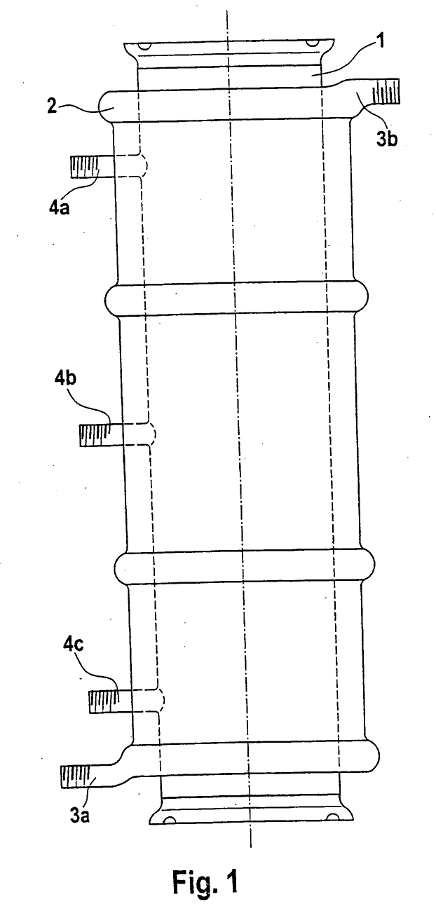

FIG. 1: A housing of the device according to the invention.

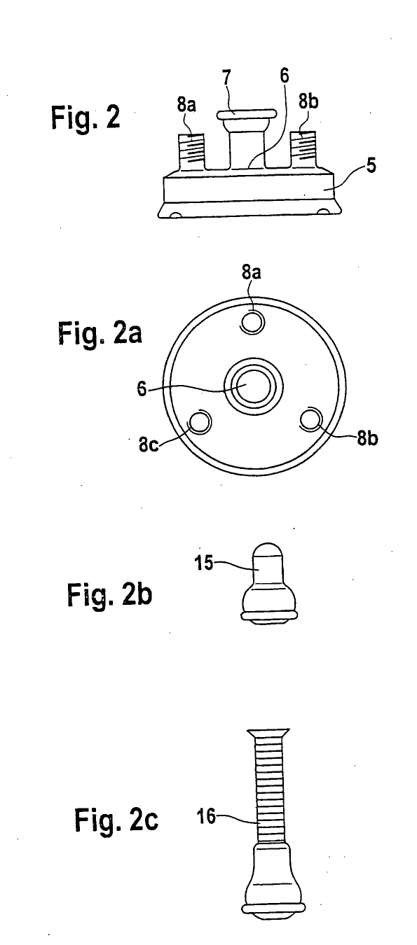

FIG. 2: An attachment piece of the housing according to FIG. 1.

FIG. 2a: A plan view of the attachment piece.

FIG. 2b: A plug for the attachment piece.

FIG. 2c: A guide sleeve for the axle.

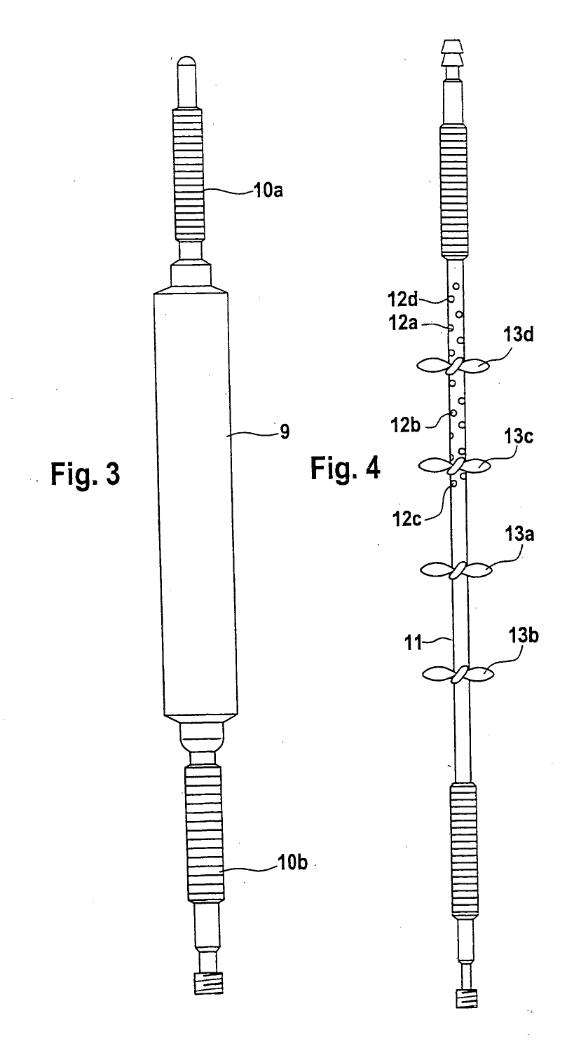

FIG. 3: A rotary roll for the device.

FIG. 4: An axle provided with propellers and having openings.

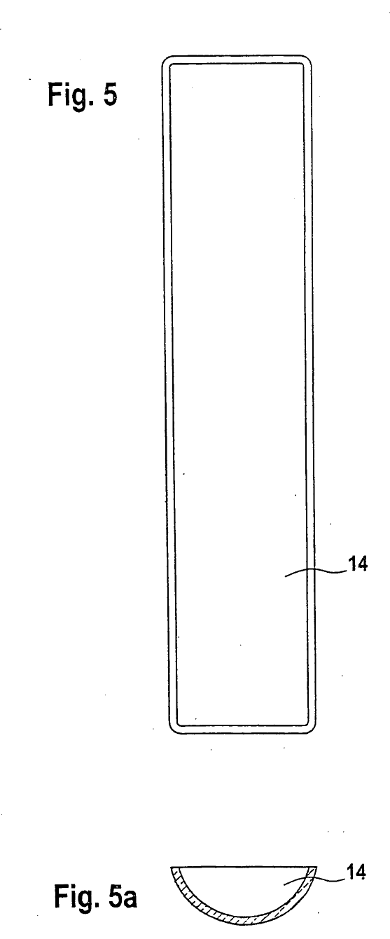

FIG. 5: A sample holder.

FIG. 5a: A cross section of the sample holder.

FIG. 1 shows a cylindrical housing 1 which is fitted with a cooling jacket 2 equipped with coolant fittings 3a, 3b. Through the cooling jacket 2 extend fittings 4a, 4b, 4c for the introduction of sensors for the measurement of, for example, temperature or air humidity and which connect the interior of the housing 1 with the external environment.

FIG. 2 shows a connecting piece 5 which is applied to an end of the housing 1. It comprises a central opening 6 which is fitted with a connecting piece 7. Furthermore, two additional connecting pieces 8a and 8b are applied to the connecting piece 5. In FIG. 2a the same reference numerals have been used to indicate the same device features as those of FIG. 2. FIG. 2b shows a plug for the connecting piece 7. In FIG. 2c a guide piece 16 has been shown for the axle of the roller in FIG. 3 and the propeller shaft in FIG. 4.

In FIG. 3 a rotary roller 9 is shown. It can be inserted into the housing 1 and extends with its ends 10a, 10b through the openings 6 of the respective connecting pieces 5.

In FIG. 4, an axle or shaft 11 has been shown with openings 12a, 12b, 12c, 12d . . . and propellers 13a, 13b, 13c. The shaft 11 can be introduced into the housing 1 as an alternative to the rotary roller 9.

In FIG. 5, a sample holder 14 has been shown which can be introduced into the housing 1.

In the following both embodiments of the device according to the invention will be described for their respective use purposes.

From the point of view of definition, the Henry constant should be understood to be the same value as the phase distribution coefficient, although this for a water-air system is commonly designated as the Henry constant.

In the case of determining the Henry constant K_{H} for the distribution of a substance in a water-air system, the sample holder 19 and the rotary roller 9 are introduced into the housing 1.

An aqueous solution of a test substance is received in the sample holder 14 to enable its phase distribution coefficient to be determined for the water/air system. The rotary roller 9 dips into the sample holder and picks up during its rotation a water film which comes into contact with the air in the chamber. Both the rotary roller 9 and the sample holder 14 as well as the housing 1 can be preferably made from glass since glass has a reduced tendency toward adsorption with respect to the compound to be investigated. In an especially preferred embodiment, the surface of the rotary roller 9 is roughened so that the wetting of the surface of the rotary roller 9 with water is especially facilitated. Through the use of the rotary roller 9, the surface area of the liquid is enlarged. A thus roughened surface of the rotary roller 9 can, for example, be produced by subjecting the surface of the rotary roller 9 to sandblasting. To determine the Henry constant K_{H}, air is fed into the housing through the connecting fittings 8a and 8b and there comes into contact with the water film disposed on the surface of the rotary roller 9. The air stream introduced through the connecting fittings 8a and 8b can be discharged at the opposite sides of the housing 1 from the device. During this process the housing 1 and the elements of the device located thereunder can be at a controlled temperature as a result of being traversed by a flow of a cooling medium through the cooling jacket 2. When a coolant is referred to in the sense of the invention, it is not mandatory that an agent is meant which only serves to cool the device but rather such a medium should have a freely selectable temperature to enable the device to be adjusted to a desired temperature. For temperature control, a thermostat can be used. As a consequence, by varying the temperature, the temperature dependency of the phase distribution coefficient can be determined. In a preferred embodiment of the invention, the air stream admitted to the housing 1 can be previously cleaned. This can be achieved by passing it through an adsorbent. As the adsorbent, for example, activated carbon can be used. The air discharged from the housing 1 which contains the sample substance from the water is collected and subjected to a quantitative chemical analysis.

In determination of the phase distribution coefficients for a soil-air system, the method is carried out in a similar say.

However, to ensure that the surface of the sample which is to come into contact with the throughflowing air does not suffer any depletion of the surface material in the substance to be investigated, preferably a preliminary investigation is carried out. For this, a sample is brought into contact with a flowing air stream over a measured time period which is advantageously as long as the measurement duration to follow and measurement is taken as to whether the concentration of the test substance in the air drops during the measurement period. Alternatively to a pretest, the concentration of the compound to be tested for in the gas phase can be established during the test by continuous measurements to determine if there is an eventual drop. Instead of water in the sample holder 14, there is soil. To determine the dependency of the air/soil distribution coefficient upon the water content of the soil, the soil can be brought into contact with a given amount of water to ensure a given moisture content. Before the beginning of the test, the chosen soil is mixed with the compound which is to be tested for and is homogenized and given a volume of water to establish a certain soil moisture content. The soil pretreated in this manner is transferred to the sample holder 14 and introduced with it into the housing 1 which is then closed. Instead of the rotary roller 9, the test device uses a shaft 11 which is equipped with the openings 12a, 12b, 12c, 12d and propellers 13a, 13b, 13c. The openings 12a, 12b, 12c, 12d can also be configured as nozzles. Preferably the openings 12a, 12b, 12c, 12d are located as shown in FIG. 3, only over a half of the shaft 11 and indeed preferably that half which is turned way from the outlet side of the housing 1 for the air. This one-sided distribution of the openings 12a, 12b, 12c, 12d has the effect that the air which is introduced by them into the housing 1 does not immediately leave the housing at the outlet side and thus the contact time is not foreshortened. The propellers 13a, 13b, 13c which are preferably uniformly spaced on the shaft 11, serve to thoroughly mix the air phase with the soil to be investigated. For the measurement of the phase distribution coefficient for the soil/air system, the shaft 11 is introduced into the housing 1, is journaled in the openings 6 and is set in rotation. The entire housing is hermetically sealed with the connection pieces against the external atmosphere. The shaft 11 with the openings 12a, 12b, 12c, 12d and the propellers 13a, 13b, 13c, 13d is set in rotation. Air is fed through the interior of the shaft 11 and emerges through the openings 12a, 12b, 12c, 12d and is uniformly distributed by the propellers 13a, 13b, 13c.

The air which is fed through the shaft 11 and the openings 12a, 12b, 12c, 12d is preferably pretreated in accordance with this test protocol. Thus foreign substances are filtered out by an adsorption agent [adsorbent]. The adsorbent can, for example, be active carbon. Furthermore, in the case of determining the phase distribution coefficients for the air/soil system, the air entering the housing 1 should be brought in a defined manner to a precise air humidity. This can be achieved, for example, by passing the air through a water-filtered washing flask. Preferably the inlet region of the shaft 11 is one connected to two washing flasks, one of which is filled with the active carbon and the other with water. By charging the incoming air with water, a drying out of the soil during the test interval is precluded. In addition in this test protocol, it is also possible to control the temperature of the housing 1 by a thermostated liquid at different temperatures. In this manner the temperature dependency of the phase distribution coefficients of a substance can be measured for the soil/air system. The air emerging at the end of the housing is again collected and subjected to a quantitative analysis. For the determination of the soil/air distribution coefficients or the Henry coefficients, it is necessary to produce a quasi-equilibrium. By this is meant that a state should be reached which does not practically deviate from an equilibrium state which can be established after a sufficiently long contact time between the air and the sample. This quasiequilibrium is achieved at throughflow rates for the air of 100 ml/min to 2500 ml/min. Naturally soil variations in the throughflow of the air are also possible with the same results. The sample which is used has, for example, a mass of 500 grams to 2500 grams since in this range the distribution coefficient of especially low volatility substances can be measured especially well. However, the emptying of the soil or generally the sample of the substances to be detected during the test interval should be avoided to prevent a fall beneath the detection limit. If one takes a factor of five above the detection limit (about 1 μg) as the basic level for permissible quantification, one can determine minimum quantities in the dry weight as follows for the various compound classes. Under these conditions for
PCB: 50 g,
PAK: 0.5 g and
Pesticide 50 g
   have been found.

From a practical view point, amounts of 500 g and more have proved to be especially preferably. This depends upon the dimensioning of the device according to the invention which should be especially easily handleable. The mass of 500 g is used for optimum handling based upon the dimensioning of the housing 1 if one assumes a layer thickness of 2 mm on the floor of the sample holder 14. The sample amounts can be, however, depending upon the size of the device, also up to 7 kg or more. With respect to the handling and quantification, sample quantities of 500 g to 1000 g have proved to be optimal. Amounts of 0.5 g to 50 g and more have been found also to be satisfactory, depending upon the sample substance. To prevent the depletion of the sample, a minimum quantity or the substance to be investigated is required and should remain with the soil or, generally in the sample and this should not fall below about 50 mg. In a less preferred embodiment, the sample can be provided in the housing 1 without a sample holder 14.

The air admitted either through the connecting piece 5 or through the openings 12 in the shaft 11, is drawn through the housing 1 by means of a pump and is collected for analytical purposes. It is significant that no air which is charged with the test substance desorbed from the water or the soil is lost. In a highly precise method, the air is collected in an adsorption tube which is selected in accordance with the substance for which the test is made and liberated in a thermodesorption system for the quantification of the adsorbed compound by a gas chromatograph coupled to that system and analyzed by a mass selective detector. Instead of a gas chromatograph, any suitable device can also be used for quantitative analysis. For example, a UV spectrometer or an NMR device can, for example, be used. The quantification of the compounds which are investigated in the soil/air or water/air compartments enable a calculated determination of the distribution coefficients.

With the method of the invention, the ambient parameters within the housing 1 can be maintained in a defined manner. Air testing is effected under quasiequilibrium conditions and the equilibrium concentration in the soil, water and air compartments are analytically determined when they exceed the analytically detectable limits when the system is used for low volatility compounds. The device according to the invention utilizes simple apparatus elements when switching one measurement of the water/air and soil/air systems is required. The system allows, therefore, the distribution coefficients or Henry constants of low volatility compounds like PCBs and PAKs to be determined. For example, PCB 28, PCB 32, polycyclic aromatic hydrocarbons (PAKs) like fluoroanthene and benzo[a]pyrene and pesticides like parathion-methyl, fenpropimorph, terbutylazin and chloropyrifons can be mentioned. The distribution coefficients can be in dependence upon temperature and/or air humidity.

It will be self-understood that with the device and method of the invention phase distribution coefficients of a sample can be determined with other gases than air. The tests carried out with the method according to the invention and the device according to the invention can be evaluated as follows:

Determining the Henry coefficient (dimensionless):
K_{H}=C_{A}/C_{W}

Where the air concentration C_{A}=the mass of the adsorbed compound [μg]/exchanged air volume in the test interval [dm³].

The concentration in aqueous solution C_{W}=mass of the dissolved compound [μg]/volume of the water introduced [dm³]
the exchanged air volume in the test interval [dm³]=the air replacement rate of the pump [dm³ min⁻¹] times the test duration [min].

The determination of the soil-air distribution coefficient K_{SW}:
K_{SW}=C_{S}/C_{A}[cm³g⁻¹]

The soil concentration C_{S}=the mass of the pesticide present [μg]/dry mass of the soil [μg].

The air concentration C_{A}=the mass of the adsorbed pesticide [μg]/replaced air volume over the test duration [dm³].

The above given formulas contain the approximation that the pesticide concentrations present in the water or in the soil at the beginning of the test remain constant over the duration of the test. The correction of these concentrations for the volatile component is not required since any error which results therefrom is negligible.

### EXAMPLE

The central component of the phase distribution chamber (SWAP-C: soil-water-air-partitioning-chamber) is comprised of a double-wall Duran glass tube (length: 1 m) with an inner diameter of 15 cm and connected to an expansion bellows and glass thread (GL18) as sketched in FIG. 1 for the connection of cooling tubes and for receiving temperature sensors or moisture sensors. The glass tube can be closed at both sides by closure caps (FIG. 2), sealing rings and rapid action closures in a pneumatic manner. In the closure caps, central ground spherical surfaces are provided for guiding the shaft (FIGS. 3 and 4). In the glass tube, a receiver (FIG. 5) for soil or water is inserted. Starting from this basic construction, the following experimental methods can be carried out.

The determination of soil/air distribution coefficients is effected through the use of a glass shaft (FIG. 4) made from boron float glass with propeller blades, is centrally positioned within the glass tubes and whose ends project outwardly through the ground spherical surfaces of the closure caps on both sides. The shaft is connected on one side of the apparatus through the use of a KPG sleeve (FIG. 2c) with a KPG stirrer. The additional power in the closure cap (FIG. 7, reference numeral 7) enables the connection of the air sampling system to the tube and which is comprised of a thermodesorption tube and a pump. The adsorption used is selected as a function of the compounds to be tested for. The power of the pump is such that it passes an air stream between 0.1 and 0.5 liters per minute through the apparatus. On the other side of the glass tube, the tube is hollow and opens by a hole in the distribution chamber for admission of air therefrom, the distribution chamber being provided with a tube connection with two washing flasks for admitting air to the chamber. A first washing flask serves for air filtration with active carbon while the second flask filled with water serves for water saturation of the air before it enters the shaft or the glass tube and thus prevents the drying out of the soil during the test interval.

Before the beginning of the test, the selected soil is mixed with the soil to be investigated and, to establish the soil moisture content, is homogenized with a given volume of water. The soil treated in this manner is transferred into the soil receptacle and the chamber is then closed. During the measurement, with the aid of a thermostat, a constant temperature is adjusted in the system and the air found in the chamber is mixed by the propeller blades. By means of the suction pump, the air sampling system draws a defined air stream through the chamber, depending upon the substance to be tested for and the compound contained in the outflowing air is filtered out with the aid of the adsorbent used. The time intervals for replacement of the adsorption tubes are selected as a function of the material to be investigated. For quantification of the adsorbed compound, a gas chromatograph with a mass selected detector (GC-MSD) coupled with a thermodesorption system is used. The quantification of the compound to be investigated in the soil and air compartment enables the calculated determination of the distribution coefficient. The determination of the water-air distribution coefficients is affected by changing the above-described configuration. Instead of the shaft provided with propeller blades, a sandblasted roller (FIG. 3) is mounted within the chamber. This roller is not provided with air holes so that the air supply is affected without the use of a second washing flask for water saturation of the air. A quasisolution of the compounds to be investigated with a known concentration is poured into the receiving container. During the test, the rotating roller dips into the solution and on the sandblasted roller surface of thin liquid film is produced with the effect of enlarging the surface area. Thermostating, air sampling and analysis are carried out analogously to the above-described method.

Taking into consideration the total volume of the distribution chamber (17.7 liters) using the minimum flow rate of the pump (0.1 liter per min⁻¹) an average residence time in the chamber of about 3 hours is obtained. The residence time at maximum throughput (1.5 liter per min⁻¹) is about 12 minutes. Starting form an approximation value of the detection limit (about 1000 pg), the degree of compaction of the soil and the soil-air distribution coefficients, duration can be determined for different compound classes which are required for the adsorption of sufficient quantities of the substances to ensure that the detection limits will be exceeded. In this manner, for polycyclic aromatic hydrocarbons (PAKs), durations of the experiments of up to 42 hours were determined to exceed the detection limits. In the case of pesticides with maximum pumping rates, durations of up to 37 hours were indicated. By varying duration and the throughput rate of the pump, the experimental conditions can be matched to the requirements and a sufficient air collection rate as possible for the respective compound class. A precondition for an effective matching is that the equilibrium setting in the soil-air system is sufficiently rapid and that the system during the test interval is in a quasiequilibrium and not traversed by an excessive flow rate which might create a noneqilibrium state. The presence of an equilibrium can be determined in a pretest by concentration measurements at uniform intervals such that constant air concentrations, depending upon the compound class are attained.

A precondition for an effective matching is that the equilibrium setting in the soil-air system is effected sufficiently rapidly and that the system during the test interval is in a quasiequilibrium and not brought to a nonequilibrium state by excessive throughflow rate. The presence of an equilibrium can be determined by pretesting through concentration measurements at regular intervals with constant air concentrations signalling the existence of the equilibrium.

In the framework of this pretest, one should not permit a depletion of the soil of the test compound by sorption of the test substance in lower soil layers. By analogy of the above-described evaluation, a determination can be made that to avoid a depletion, a certain mass of soil should be exceeded for tests with the particular distribution chamber, for example, the minimum dry mass of soil in the soil-receiving vessel should be 500 g. Corresponding detection limits and test time frames can also be determined for the determination of water-air distribution coefficients.

1. A device for determining phase distribution coefficients comprising a housing, an inlet and an outlet for gas, characterized in that,
it encompasses the following features:
a rotary roller (9) and openings (8a, 8b, 8c) located on opposite sides of the housing (1) for gas entry and gas discharge and/or
a shaft (11) which is provided with openings (12a, 12b, 12c, 12d) through which the gas inflow into the housing and on which propellers (13a, 13b, 13c, 13d) are mounted,
as well as openings (6) on opposite sides of the housing (1) for receiving the rotary roller (9) or the shaft (11).

2. The device according to claim 1, characterized in that, the housing (1) extends longitudinally along the shaft (11) or the rotary roller (9).

3. The device according to claim 1, characterized in that, the rotary roller (9) has a roughened surface.

4. The device according to claim 1, characterized in that, the rotary roller is comprised of glass.

5. The device according to claim 1, characterized in that, the propellers (13a, 13b, 13c) on the shaft (11) are uniformly spaced.

6. The device according to claim 1, characterized in that, the openings (12a, 12b, 12c) of the shaft (11) are provided on the half of the shaft (11) which is turned toward the gas outlet (7).

7. The device according to claim 1, characterized in that, a sample holder (14) is located in the housing (1).

8. The device according to claim 1, characterized in that, it encompasses a pump which displaces the gas through the housing (1).

9. The device according to claim 1, characterized in that, the gas outlet (8a, 8b, 8c) has an analyzing device downstream thereof.

10. The device according to claim 9, characterized in that, the analysis device encompasses an adsorption tube and thermodesorption device.

11. The device according to claim 9, characterized in that, the analysis device encompasses a gas chromatograph.

12. The device according to claim 1, characterized in that, the housing (1) has a washing flask upstream thereof.

13. The device according to claim 1, characterized in that, the housing (1) has a container with an adsorbent upstream thereof.

14. The device according to claim 13, characterized in that, the adsorbent is active carbon.

15. The device according to claim 1, characterized in that, it has at least one connection (41, 4b, 4c) for connecting measuring instruments.

16. The device according to claim 1, characterized in that, it is equipped with a temperature and/or moisture measuring unit which measures values in the interior of the housing (1).

17. A method of determining this distribution coefficients of substances in a liquid/gas system or a solid/gas system, characterized in that a gas stream of known volume is brought into contact with a probe containing the substance and conducted over an adsorbent, after which the adsorbent is thermodesorbed and desorbed material is subjected to a quantitative chemical analysis.

18. The method according to claim 17, characterized in that, an aqueous solution of the substance is used in the sample.

19. The method according to claim 18, characterized in that, soil containing the substance is used as the sample.

20. The method according to claim 17, characterized in that, the sample has a mass in the range of 0.5 g to 7 kg.

21. The method according to claim 20, characterized in that, the sample has a mass of 500 g to 2.5 kg.

22. The method according to claim 18, characterized in that, the surface of the liquid is increased while the gas is passed over it.

23. The method according to claim 17, characterized in that, the gas stream before being guided over the sample is freed from impurities.

24. The method according to claim 23, characterized in that, the gas stream is passed over active carbon.

25. The method according to claim 17, characterized in that, the air stream prior to being passed over the sample is charged with moisture.

26. The method according to claim 17, characterized in that, the sample has a defined moisture content.

27. The method according to claim 17, characterized in that, the gas and the sample are temperature-controlled to a desired temperature.

28. The method according to claim 17, characterized in that, the gas stream passed over the sample after thermoadsorption is quantitatively analyzed in a gas chromatograph.

29. The method according to claim 17, characterized in that, the gas stream has a speed of 100 ml/min to 2500 ml/min.

30. The method according to claim 17, characterized in that, air is used as the gas.
